(19) United States
(12) Patent Application Publication
(10) Pub. No.: US 2026/0028752 A1
(43) Pub. Date: Jan. 29, 2026

(54) METHODS AND COMPOSITIONS FOR SPATIALLY-RESOLVED SINGLE CELL SEQUENCING

(71) Applicant: Scale Biosciences, Inc., Pleasanton, CA (US)

(72) Inventors: Frank STEEMERS, Encinitas, CA (US); Felix SCHLESINGER, San Diego, CA (US)

(21) Appl. No.: 19/349,447

(22) Filed: Oct. 3, 2025

Related U.S. Application Data
(63) Continuation of application No. PCT/US2024/019242, filed on Mar. 8, 2024.
(60) Provisional application No. 63/457,559, filed on Apr. 6, 2023.

(51) Int. Cl. C40B 40/02; C12N 15/113; C12Q 1/6869; C40B 50/06; C40B 70/00

(52) U.S. Cl. CPC C40B 40/02; C12N 15/113; C12Q 1/6869; C40B 50/06; C40B 70/00

(57) ABSTRACT

Provided herein, among other things, is a method for spatial single cell analysis. In some embodiments, the method may comprise: obtaining a cellular sample comprising nucleic acid molecules, binding spatial tags to the sample, removing any unbound or unreacted spatial tags, determining the location and identity of the spatial tag, dissociating the sample into single cells, performing single cell sequencing, determine the spatial barcode for each single cell, and assigning the single cell sequencing reads to its spatial location.

### CROSS-REFERENCING

This application is a continuation of International Application No. PCT/US2024/019242, filed on Mar. 8, 2024, which claims the benefit of U.S. provisional application Ser. No. 63/457,559, filed on Apr. 6, 2023, which applications are incorporated by reference herein.

### BACKGROUND

Single cell sequencing has become a standard tool for studying how genes are regulated, cellular states and cellular functions at the single cell level. However, transcription in individual cells is influenced by their localization within a particular tissue. As such, in order to gain a more complete understanding of a cell, one should obtain information about gene expression in individual cells in their morphological context.

Current methods for analyzing single cells and their respective analytes in tissue sections are limited. For example, in situ hybridization provides a way to analyze transcripts in a tissue section, but the number of transcripts that can be analyzed in one experiment is rather limited. Next-generation sequencing approaches have the potential to provide a solution to this problem. However, most sequencing-based approaches require compartmentalization of single cells, which removes the spatial context (e.g., morphologically, context of the cells in the tissue etc.). Other sequencing-based platforms rely transferring RNA from a tissue section to a microarray (see, e.g., Bergenstråhle et al. BMC Genomics (2020) 21: 482). Such array-based methods have low capture efficiency, relatively low resolution, and a certain amount of spatial information is lost when the RNA molecules diffuse from the tissue to the array. As such, array-based methods are unsatisfactory for a number of applications. Additionally, the cost and complexity of making the barcoded arrays is high and has limited spatial resolution.

Other spatial in situ methods combine spatial tagging and in situ assays. Such methods are complex as one simultaneously need to develop spatial tagging and in situ assay for an analyte of interest, making this a challenging and complex system, including the spatial high-resolution requirements, has limited multiplexing capabilities, and often ignore the 3D structure of a tissue sample.

This disclosure describes methods that utilize single cell sequencing and spatial tagging approaches to provide single cell sequencing information with spatial information. The methods provide spatially-addressable barcodes, comprising spatial tags, to a cellular sample and use orthogonal methods to determine the location and identity of the spatial tags in situ. The cells of the sample are dissociated while maintaining the association of each cell with the associated spatial tags, and individual cells and associated spatial tags are analyzed together using single cell sequencing. This approach provides a way to resolve single cell profiles and their spatial information across various assay modalities. In one embodiment, the spatial tags are randomly added in a single step to the cellular sample by adding a highly diverse spatial tag pool in a way that is limiting resulting in sub-sampling of barcodes such that each cell receives a unique spatial tag or combination of spatial tags. The spatial tags can be detected and identified in the spatial context using a variety of methods including microscopy and fluorescence decoding (i.e. decoding Randomly Ordered DNA Arrays Genome Res. 2004 May: 14(5): 870-877). The cells are dissociated and individual cells and associated spatial tags are analyzed through single cell sequencing (e.g., single cell compartmentalization or combinatorial single cell sequencing and barcoding each cell and associated spatial tag(s) uniquely). After sequencing the spatial tags and single cell libraries, the spatial tags can be determined for each cell and subsequently the sequencing reads for each cell can be mapped to a site in the tissue section using the spatial tags. This method can be used to provide profiles for various analytes of the single cells that are in or on a tissue section using many known and future single cell sequencing methods, does not require complex in situ assays or barcoded arrays, and thus solves the problems discussed above.

### SUMMARY

Provided herein, among other things, is a method for adding spatially addressed nucleic acid barcodes (spatial tags) in or on a cellular sample in situ. In some embodiments the method may comprise: obtaining a cellular sample comprising nucleic acid molecules, staining the cells or contents of the cells with spatial tags (e.g. randomly distributed barcode or barcodes with an assay handle (‘assay handle can be a common sequence to enable readout of the spatial tag using single cell sequencing), determine the identity and location of the spatial tags on or in the cellular sample, generating a single cell or nuclei suspension of the cellular sample while maintaining the association with the spatial tag(s), and analyzing the analytes and spatial tag(s) of the single cells through single cell sequencing (e.g. compartmentalization of single cells and uniquely barcoding the cells and spatial tag(s)). The sequenced single cell barcodes and spatial tags provide single cell data in the spatial context of the cells in the cellular sample.

The method can, for example, be used to randomly add spatially-addressable barcodes to cells or nuclei of a cellular sample through specific or non-specific binding. In this embodiment, the spatial tag's locations and identity may be determined through sequential hybridization and fluorescent imaging. The single cells and associated spatial tags may be analyzed through single cell sequencing. This process adds the same single cell barcode to analytes of interest and the associated spatial tag(s) from the same cell. The sequenced products and the single cell sequences can be mapped to a physical position on the sample using the spatial tags.

### BRIEF DESCRIPTION OF THE FIGURES

The skilled artisan will understand that the drawings described below are for illustration purposes only. The drawings are not intended to limit the scope of the present teachings in any way.

FIG. 1 illustrates some of the principles of an embodiment of the present method.

FIG. 2A example workflow to obtain single cell omics data with spatial information illustrates how a cellular sample can be tagged with random spatial tags.

FIG. 2B example of decoding the spatial tag to determine its identity and spatial location illustrates examples of single cell sequencing libraries made for RNA and the spatial tag after the method.

FIG. 2C example workflow to obtain single cell omics data with spatial information.

FIG. 2D example of a spatial tag sequence.

FIG. 3, example of sequencing libraries generated for the spatial tag and the associated single cell libraries.

FIG. 4 illustrates example workflow how sequencing libraries for RNA and the spatial tag can be prepared through simultaneously single cell barcoding the cell and spatial tag in the same compartment.

FIG. 5 illustrates an example of a design for the spatial tag.

### DEFINITIONS

Unless defined otherwise herein, all technical and scientific terms used in this specification have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N. Y. (1991) provide one of ordinary skill in the art with the general meaning of many of the terms used herein. Still, certain terms are defined below for the sake of clarity and ease of reference.

As used herein, “analyte”, “target analyte”, “cell/cellular analyte”, or “sample analyte” refers to any specific species of molecule, or its surrogate or derivative, or its characteristics that are identified and quantified within a cell or sample. This encompasses a wide range of molecular and macromolecular types, including nucleic acids (DNA and RNA), proteins, lipids, carbohydrates, metabolites, and ions, among others. Assays can be tailored to detect and measure the presence, quantity, and specific properties of an analyte, including its variants, modifications, and other unique features. For instance, when examining an mRNA or DNA molecule, relevant characteristics may include the gene from which it originates, any sequence variations (like mutations or single nucleotide polymorphisms), different forms resulting from splicing, and chemical modifications such as methylation. Thus, an analyte assay can determine both the identity of a molecule and its quantity, along with detailed attributes.

A “hash” tag is a molecular tag that is associated with both a single cell barcode sequence and a sequence denoting one or more attributes of a single cell such as its sample of origin, the spatial location of the cell, a perturbation of the cell, or any other information (historical or otherwise) associated with a particular cell. The single cell barcode sequence associated with the hash is also informatically associated with the single cell barcode sequence used in the single cell analyte assays. Exemplary uses of “hashing” include sample multiplexing (and doublet removal) in single cell assays (Stoeckius et al. Genome Biology 2018 19: 224; Gaublomme Nature Communications 2019 10: 2907), single cell spatial hashing (Srivatsan et al. Science 2021 373: 111-117; Russell et al. Nature volume 625, pages 101-109 (2024), single cell chemical perturbation hashing (SCIENCE 2019 367: 45-51), cell-cell interaction hashing (Li et al. Cell Rep Med. 2023 4: 101121), single cell CRISPR screens hashing (Replogle et al. Cell 2022 185: 2559-2575), and any of other attributes associated with single cell experiments. A hash tag and related methods may enable one or more attributes including: (i) sample identification, (ii) single cell identification (multiplet removal), or (iii) multiplexing and super-loading on single cell sequencing platforms.

A “spatial hash tag” is a molecular tag or combination of tags that uniquely identifies the spatial location of a single cell, or part of the single cell, along with single cell barcode sequence associated with the analytes. Exemplary “spatial hash tags” include single cell spatial hashing as described by Srivatsan et al., 2021 and Russel et al., 2023 (Srivatsan et al. 2021; Russell et al. 2023). The spatial hash tag may bind or have affinity to cellular structures, membrane, analytes, analyte derivatives, any molecule or organelle of the cell. The term “hashing” refers to the process of attaching a “hash tag” to a cellular sample, wherein the “hash tag” is attached to a molecular component of the cell, which may or may not be an analyte. Moreover, the “hash tag” is associated with a single cell barcode to which the cellular analytes are also associated, and the “hash tag” is not in a one-to-one ratio with the analytes, but rather a small subset of the resultant single cell library.

As used herein, the term “spatially addressed” and “spatially addressable” and “spatial” refer to sequences that can be mapped to a site or position on a sample, e.g., by x-y-z coordinates.

As used herein, the term “nucleic acid barcode” refers to a sequence of nucleotides that is appended onto one or more target nucleotides. A nucleic acid barcode can be at least 4nucleotides, e.g., 4-20 nucleotides, in length.

As used herein, the term “spatially addressed nucleic acid barcode” refers to sequences of nucleotides that are appended onto one or more target polynucleotides, where the sequence appended onto each target polynucleotide indicates a position on the sample, e.g., by x-y-z coordinates. A sample that contains spatially addressed nucleic acid barcodes can be subdivided into multiple areas, where each area is associated with a different barcode sequence.

As used herein, the term “cellular sample” is intended to include samples are made by, e.g., growing cells on a planar surface, samples that are made by depositing cells on a planar surface, e.g., by centrifugation, and samples that are made by cutting a three-dimensional object that contains cells into sections and mounting the sections onto a planar surface, i.e., producing a tissue section. The surface upon which a sample may be mounted may be, e.g., glass, metal, ceramic, plastic, etc.). If the sample is fixed, it may be fixed using any number of reagents including formalin, methanol, paraformaldehyde, methanol:acetic acid, glutaraldehyde, bifunctional crosslinkers such as bis(succinimidyl)suberate, bis(succinimidyl)polyethyleneglycol etc. A section (e.g., a cryosection) of a tissue sample (e.g., of a fresh frozen tissue sample) that has a thickness in the range of 1-50 μm (e.g., in the range of 1-5 μm or 5-20 μm) is an example of a cellular sample, although there are many alternatives. In some embodiments the cells in the sample may be fixed and/or permeabilized, e.g., using a detergent or a solvent.

As used herein, the term “tissue section” refers to a piece of tissue that has been obtained from a subject and mounted on a planar surface, e.g., a microscope slide.

As used herein, the term “formalin-fixed paraffin embedded (FFPE) tissue section” refers to a piece of tissue, e.g., a biopsy sample that has been obtained from a subject, fixed in formaldehyde (e.g., 3%-5% formaldehyde in phosphate buffered saline) or Bouin solution, embedded in wax, cut into thin sections, and then mounted on a microscope slide.

As used herein, the term “spatial tag, barcode agent, or barcode” As used herein, the term “spatial tag or barcode agent” refers to a polynucleotide with any suitable length, e.g., a nucleic acid molecule of about 2 bases to about 100 bases, including any integer including 2 and 100 and in between, that comprises identifying information for its spatial cellular location. A “spatial tag, barcode agent, or barcode tag” may also be made from a “sequenceable polymer” (see, e.g., Niu et al., 2013, Nat. Chem. 5: 282-292; Roy et al., 2015, Nat. Commun. 6: 7237; Lutz, 2015, Macromolecules 48: 4759-4767; each of which are incorporated by reference in its entirety). In some embodiments, the spatial tag is a hash tag, wherein the tag is attached to the cellular sample via molecular attachment points that may or not be the analyte, and generally not in a one-to-one ratio with the analyte, and is associated with the analyte via the single cell barcode. A barcode tag may comprise an encoder sequence, which is optionally flanked by a primer. A spatial tag may also contain an optional UMI and/or primer, common sequence, decode sequence, sequencing primer, handle for assays to ensure the spatial tag is readout using sequencing. A spatial tag may be single stranded or double stranded. A double stranded spatial tag may comprise blunt ends, overhanging ends, or both. A spatial tag(s) may refer to the spatial tag(s) that is directly attached to a binding agent, to a complementary sequence hybridized to the spatial tag(s) directly attached to a binding agent. In certain embodiments, a spatial tag may further comprise a spacer or barcode, a unique molecular identifier, a universal priming site, sequencing primer, or any combination thereof. The spatial tag or spatial tags can consist of a pool of barcodes. The spatial tag pool can contain 1 or more barcodes. The barcode pool complexity can range from 1 to millions, to trillions or more different barcodes. In some embodiments, a majority of barcode molecules in the pool are unique. The spatial tag can be part of a complex or branched molecular structure to which oligonucleotides can hybridize or bind. In some embodiments, the spatial tag is introduced using in situ hybridization. In some embodiments, a precursor is introduced first that enables the binding of the spatial tag e.g., Ab-oligo conjugate, oligonucleotide etc. In some embodiments, the spatial tag contains or binds to an affinity binder e.g., antibody etc. Any material or molecule that be spatially identified through optical, electrochemical, biochemical, or chemical or other means, and can be readout through DNA or protein sequencing can be used. In the preferred embodiment, the readout of the spatial tag is non-destructive to determine the spatial location and spatial barcode information. In some embodiments, spatial tags are randomly distributed over the cellular samples such that each cell, nuclei, and/or sub-components or compartments of the cell receives a unique spatial tag or barcode combination. In some embodiments, the spatial tag, decode sequence or derivative can be amplified, multiplied, or replicated to improve detection. The spatial tag can contain a decode sequence to determine the spatial barcode. In some cases, the decode sequence or barcode or combination of barcodes are the same. In some cases, the spatial tag or decode sequence consists of multiple sub-sequences or barcodes. In some cases, the relationship between the decode sequence and the spatial barcode is known.

As used herein, the term “single cell barcode or single cell barcode tag” As used herein, the term “single cell barcode” refers to a polynucleotide or combination of polynucleotides with any suitable length, e.g., a nucleic acid molecule of about 2 bases to about 100 bases, including any integer including 2 and 100 and in between, that comprises identifying information for each single cell. The single cell barcode can contain one or more subunits of oligonucleotides (see example, WO 2012/106385 A2). The single cell barcode tag is unique from cell to cell. A “single cell barcode or single cell barcode agent or barcode tag” may also be made from a “sequenceable polymer” (see, e.g., Niu et al., 2013, Nat. Chem. 5: 282-292; Roy et al., 2015, Nat. Commun. 6: 7237; Lutz, 2015, Macromolecules 48: 4759-4767; each of which are incorporated by reference in its entirety). A tag may comprise a random sequence, which is optionally flanked by a primer. A barcode tag may also contain an optional sample barcode, primer, or sequencing primer. A barcode tag may be single stranded or double stranded. A double stranded coding tag may comprise blunt ends, overhanging ends, or both. In certain embodiments, a single cell barcode tag may further comprise a unique molecular identifier, a universal priming site, or any combination thereof. In some cases, the spatial tag has a branched structure, assembly of multiple components, bound through a molecular assembly, or as a single structure, to which multiple decoders can hybridize to improve detection of the spatial tag. In some embodiments, the spatial barcode is different than the decode sequence. In some embodiments, the spatial barcode and the decode sequence are the same.

As used herein, the term “removing” refers to any action that results of the elimination of a compound. Removing may include degrading, inactivating, or washing away, or any combination thereof. 20

As used herein, the term “5′ or 3′ tail”, in the context of a tailed oligonucleotide, refers to a 5′ or 3′ part of an oligonucleotide that is not complementary to a target and does not hybridize to the target that the 3′ or 5′ hybridizes to, respectively. A tail can be as long as needed, e.g., in the range of 20-100 bases, as desired.

As used herein, the term “oligonucleotide” refers to a multimer of at least 2 nucleotides, e.g., at least 5, at least 10, at least 15 or at least 30 nucleotides. In some embodiments, an oligonucleotide may be in the range of 15-200 nucleotides in length, or more. Any oligonucleotide used herein may be composed of G, A, T and C, or bases that are capable of base pairing reliably with a complementary nucleotide. In some embodiments, an oligonucleotide may additionally contain one or more “universal” bases that can base pair with any of G, A, T and C. Universal bases include 2′-deoxyinosine 2′-deoxynebularine, 3-nitropyrrole 2′-deoxynucleoside and 5-nitroindole 2′-deoxynucleoside, although others are known. Oligonucleotides may be synthetic or may be made enzymatically, and, in some embodiments, are 30 to 150 nucleotides in length. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example.

The term “decoding’ refers to a process to determine the identity and location of the spatial tag. The process can be optical, electrical, chemical and can be a single cell step, or iterative steps to determine the identity and location of the spatial tag or decode sequence. In some cases, the decoding involves hybridization or affinity binding of fluorescent decoders, removing unbound decoders, imaging the decoders. In some cases, decoding of the spatial tags occurs through sequential hybridization of fluorescent decoders, removing unbound decoders, imaging, removing the fluorescent decoders, and repeating the process multiple times with different sets of decoders to determine the identity and location of the spatial tags. In some embodiments, the spatial tags are decoded using sequencing. Technologies related to single molecule FISH can also be used for the detection and identification of the spatial tags (e.g., single-molecule Fluorescence in situ Hybridization (smFISH) for RNA Detection in Adherent Animal Cells). Haimovich et al. Bio Protoc. 2018 Nov. 5; 8(21). In some embodiments, z-probes, pre-amplifiers or amplifiers (amplification schemes) can be used to visualize and detect the spatial tags e.g. using single molecule mRNA fluorescent in situ hybridization (RNA-FISH) to quantify mRNAs in individual murine oocytes and embryos Xie et al. Scientific Reports volume 8, Article number: 7930 (2018) and references cited herein. In some embodiments, not all spatial tags present in the sample are decoded or analyzed in single cell sequencing. The spatial tags or combination of all decoded spatial tags are unique or substantially unique from cell to cell. Other methods of introducing spatial tags including various methods of cell hashing and analyte tagging as previously described (Hashing with barcoded antibodies enables multiplexing and doublet detection for single cell genomics, Stoeckius et al. Genome Biology vol. 19, Article number: 224 (2018) or Comparative analysis of antibody-and lipid-based multiplexing methods for single-cell RNA-seq Mylka et al. Genome Biology volume 23, 55 (2022) and reference cited herein). Various spatial methods have been described and can be used for the visualization and identification of the spatial tags described in this invention (for example, see Museum of spatial transcriptomics Moses et al. Nature Methods volume 19, pages 534-546 (2022)). For example, the nCounter Analysis System from nanostring can detect 800+ target analytes (see, Goytain et al. NanoString nCounter Technology: High-Throughput RNA Validation Methods Mol Biol 2020; 2079: 125-139. In some cases, this process can be repeated with different pools of decoders.

The term “binding” or “bind” refers to a process in which a molecule e.g., nucleic acid strand binds to an analyte of a cell or part of the cell, or cell compartments, structure, backbone, organelle etc. The binding can be specific, non-specific, or random. The binding can be maintained during cell lysis, cellular sample dissociation (e.g., tissue et), or single cell or nuclei suspension formation. The binding can be to cellular analytes or non-analytes (e.g., cell wall, cell structure etc.). In some cases, different spatial tags bind to different molecules, analytes, or cellular compartments, cell structures, cells or nuclei of the same cell of the cellular sample.

The term “hybridization” or “hybridizes” refers to a process in which a nucleic acid strand anneals to and forms a stable duplex, either a homoduplex or a heteroduplex, under normal hybridization conditions with a second complementary nucleic acid strand and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions includes hybridization at about 42C in 50% formamide, 5× SSC, 5× Denhardt's solution, 0.5% SDS and 100 μg/ml denatured carrier DNA followed by washing two times in 2× SSC and 0.5% SDS at room temperature and two additional times in 0.1×0 SSC and 0.5% SDS at 42° C.

The term “sequencing”, as used herein, refers to a method by which the identity of at least 2 consecutive nucleotides (e.g., the identity of at least 5, at least 10, at least 50 or at least 100 or more consecutive nucleotides) of a polynucleotide are obtained.

The term “next-generation sequencing” refers to the so-called parallelized sequencing-by-synthesis or sequencing-by-ligation platforms currently employed by, e.g., Illumina, Life Technologies, BGI Genomics (Complete Genomics technology), PacBio, Oxford Nanopore, Ultima Genomics, and Roche etc.

The term “duplex,” or “duplexed,” as used herein, describes two complementary polynucleotides that are base-paired, i.e., hybridized together.

The terms “determining,” “measuring,” “evaluating,” “assessing,” “assaying,” and “analyzing” are used interchangeably herein to refer to forms of measurement and include determining if an element is present or not. These terms include both quantitative and/or qualitative determinations. Assessing may be relative or absolute.

The term “ligating”, as used herein, refers to the enzymatically catalyzed joining of the terminal nucleotide at the 5′ end of a first DNA molecule to the terminal nucleotide at the 3′ end of a second DNA molecule.

The terms “plurality”, “set” and “population” are used interchangeably to refer to something that contains at least 2 members. In certain cases, a plurality may have at least 10, at least 100, at least 100, at least 10,000, or at least 100,000 members.

A “primer binding site” refers to a site to which an oligonucleotide hybridizes in a target polynucleotide or fragment. If an oligonucleotide “provides” a binding site for a primer, then the primer may hybridize to that oligonucleotide or its complement.

The term “strand” as used herein refers to a nucleic acid made up of nucleotides covalently linked together by covalent bonds, e.g., phosphodiester bonds.

The term “extending”, as used herein, refers to the extension of a nucleic acid by the addition of nucleotides using a polymerase or by a chemical reaction.

As used herein, the term “splint” refers to an oligonucleotide that hybridize to the ends of two other polynucleotides.

The term ‘UMI’ as used herein refers to a unique molecular identifier to make a library unique or substantially unique.

The term “spatially associated cells” is intended to refer to a sample in which the cells are held in place relative to one another, e.g., by an extracellular matrix. Tissue sections and pieces of tissue are examples of samples that contain spatially associated cells. A suspension of cells does not contain cells are held in place relative to one another. As such, a suspension of cells does not contain spatially associated cells. A sample containing spatially associated cells can be substantially planar (e.g., a tissue section or a sheet of cultured cells, etc.) or three-dimensional, e.g., a piece of tissue or a whole tissue. Other definitions of terms may appear throughout the specification.

### DETAILED DESCRIPTION

Provided herein, among other things, is a method for spatially labeling cells using spatial tags in or on a cellular sample in situ. In some embodiments, the method may be performed on a cellular sample (e.g., a tissue section) comprising nucleic acid molecules. As will be explained in greater detail below, the sample may be made in a variety of different ways, e.g., by hybridizing, specific or non-specific binding, adsorption, one or more spatial tags to the sample. Depending on how the method is implemented the spatial tag or barcodes may bind to an analyte or cell structure, synthesized, or coupled to DNA, RNA, cDNA, oligonucleotides, and synthetic DNA e.g., oligonucleotides, as desired. The spatial tag can be optionally labeled (i.e., dye, fluorescent, chemifluorescent, luminescence, etc.). The label can provide the identity of the spatial tag, and the spatial tag can be detected together with single cell sequencing. The spatial tag will receive the same single cell barcode as the analytes or derivatives for that cell. Once the spatial tag is determined for each cell during single cell sequencing, the single cell sequencing libraries.

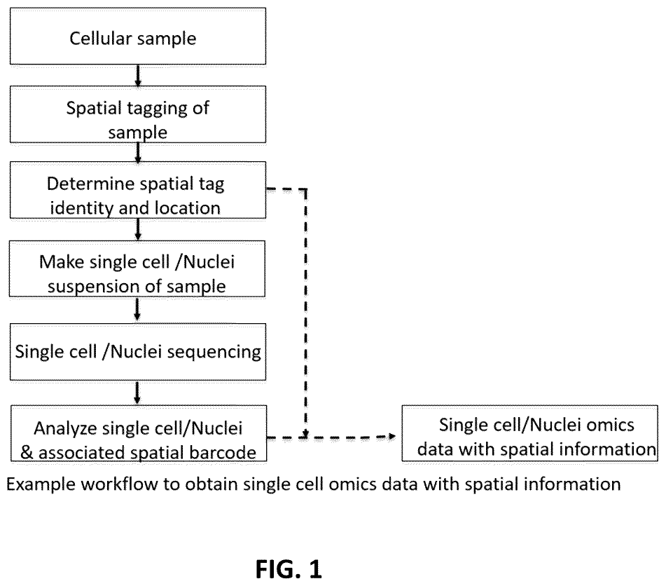

FIG. 1 illustrates some principles of the method. As illustrated in FIG. 1, the method may comprise binding the spatial tag pool in or on a cellular sample. The ideal conditions are that each cell in the cellular sample receives a unique spatial tag or spatial tag combination. As illustrated, this step may be implemented by binding or adsorption of the spatial tag. However, other methods may be used as outlined in this invention. The current method distributes spatial tags to the cellular sample. In principle, any size area of the cellular sample can be used, or regions of interest (ROI) can be used.

In some embodiments, the method may comprise adding spatial tags to cells (labeling). In these embodiments, the method may comprise: (a) distinguishably labeling at least some of the cells of a sample containing spatially associated cells; (b) analyzing the sample to identify (i) the positions of cells in the sample and (b) the labels that are bound to the cells; and (c) dissociating the sample into individual cells without disassociating the tags from the cells, to obtain a cell suspension in which the locations of some of the cells in the sample of (a) is known. If one analyzes the cells using a cell-by-cell method (e.g., single cell sequencing), then data obtained from the cells can then be mapped to a location in the sample. The labeling may be done by randomly distributing the nucleic acid tags across and/or through the sample such that the combination of tags that label a particular cell is determined by chance. In other words, the tags may be deposited as a mixture to an area that contains many cells, the tags distribute themselves throughout the sample randomly, and the positions of cells in the sample and (b) the labels that are bound to those cells is determined afterwards. In contrast to methods in which cells are deterministically labeled with spatial barcodes derived from spatially patterning of spatial barcodes, the above method distinguishably labels cells with spatial barcodes such that (i) the positions of cells in the sample and (b) the labels that are bound to the cells are both unknown at the time of labeling.

In other embodiments, the tags are distributed to specific sites onto the cellular sample using ordered or randomly-ordered arrays containing tags. Examples, but not limited, include spatial transcriptomics using multiplexed deterministic barcoding in tissue Wirth et al. Nature Communications vol 14, 1523 (2023), slide-tags enables single-nucleus barcoding for multimodal spatial genomics Russell et al. Nature volume 625, pages 101-109 (2024), and Slide-seq: A scalable technology for measuring genome-wide expression at high spatial resolution Stickels et al. Science, 2019, Vol 363, Issue 6434, pp. 1463-1467. In some of these examples, the tags can be transferred to the cellular sample using arrayed tags e.g., Embryo-scale, single-cell spatial transcriptomics Srivatsan et al. Science (2021).

The method may be implemented in a variety of different ways. For example, the method may comprise: (a) labeling a sample containing spatially associated cells with a population of nucleic acid tags, wherein at least some of the cells in the sample become distinguishably tagged by the nucleic acid tags; (b) analyzing the sample to identify: (i) the positions of cells in the sample; and (ii) on a cell-by-cell basis, the nucleic acid tags that are bound to the cells of (b)(i); and (c) dissociating the sample into individual cells without disassociating the tags from the cells, to obtain a cell suspension in which at least some of the cells are distinguishably tagged.

In some embodiments, the analyzing step (b) may comprise identifying the labels that are bound to the cells and, optionally, the positions of cells in the sample.

In some embodiments, the method may further comprise: (d) on a cell-by-cell basis analyzing: (i) the labels associated with associated with the cells of the cell suspension and (ii) analytes that are in or on at least some of the cells in the cell suspension. In these embodiments, the method may further comprise (e) mapping data obtained in (d)(ii) to a position identified in (b).

In some embodiments, the method may further comprise: (d) on a cell-by-cell basis: (i) analyzing analytes in or on the cells of the cell suspension to produce single cell data; and (ii) sequencing the nucleic acid tags that are associated with the cells of the cell suspension, and in some cases: (e) on a cell-by-cell basis, mapping the single cell data of (d)(i) to a position in the sample using the tag sequences of (d)(ii). In these embodiments, step (b) may further comprise (iii) recording the positions of distinguishably tagged cells in the sample based on (i) and (ii); and in step (e) the single cell data for cells that distinguishably tagged are mapped using the positions recorded in (b)(iii).

In any embodiment, positions may be defined by two- or three-dimensional coordinates of individual cells in the sample. The positions of the cells may be defined by their boundaries, their geometric centers, or their nuclei, for example.

The population of nucleic acid tags used in the method may comprise at least 100, at least 500, at least 1,000, at least 5,000 or at least 10,000 barcoded oligonucleotides, which, in some embodiments, are attached to a binding agent such as an antibody. In these embodiments, each barcoded oligonucleotide may comprise a defined (i.e., not random) barcode sequence (typically of 8-30 nucleotides in length) that is different from the barcode sequences of the other barcoded oligonucleotides. In embodiments in which the nucleic acid tags are attached to antibody, the antibody may bind to a protein that is constitutively expressed in or on the cells. “Nucleic acid tags” may be referred to as “spatial tags” in other parts of this disclosure.

In some embodiments, the population of nucleic acid tags may be distributed across and/or through the sample randomly, meaning that the number of molecules of a nucleic acid tag that bind to a particular cell as well as the identity of the tags of the molecules that bind to a cell are not pre-determined. In some embodiments, the average number of tag molecules that bind to cell may be in the range of 2-100, e.g., 2-50 or 2-10. Illustrated by example, seven cells can become distinguishably labeled using three labels, X, Y and Z. In this hypothetical example, some cells of the sample may bind to only one tag molecule (e.g., having tag X, Y or Z), other cells may bind to exactly two tag molecules (e.g., having tags X and Y, X and Z, or Y and Z), and other cells may bind to exactly three molecules (e.g., having tags X, Y and Z) and so on. In this example, a cell that is bound to only one tag molecule may be distinguishably labeled because that is the only cell that is bound to that molecule on its own (e.g., it is the only cell associated with tag X alone, i.e., without Y or Z); a cell that is bound by two tag molecules (e.g., having tags X and Y) may be distinguishably labeled because that is the only cell that is bound by tags molecules X and Y (and not tag Z). Finally, a cell that is bound to all three tag molecules may be distinguishably labeled because it is the only cell that is bound to all three tags but not any others (e.g., it is the only cell associated with tags X, Y and Z). In this example seven cells can, in theory, be distinguishably labeled using only three tags. In practice, more tags are used and, as such, several hundred thousand or millions of cells can be distinguishably labeled using the same principle. The complexity of the population of tag molecules required for tagging a sample can be readily determined. In some embodiments, the cells are distinguishably labeled by a combination of nucleic acid tags. As such, the term “distinguishably tagged” refers to cells that are distinguishable from all other cells by the tags to which they are bound. A cell may be distinguishably labeled by a single tag that is unique to the cell or a unique combination of tags (i.e., a combination of tags that is not bound to other cells).

In the method, the positions of cells in the sample the nucleic acid tags that are bound to the cells may be analyzed using any of a variety of methods, e.g., by hybridizing fluorescent probes to the barcode sequence and then detecting a signal. The sample may also be stained, e.g., using a cytological stain so that other features of the cells or extracellular matrix can be observed. Ideally, the tags should be detected using a method that single molecule resolution (so that single binding events can be optically resolved from one another), e.g., smFISH, seqFISH, or the like (see, e.g., Tingey et al, Cells 2022, 11, 3079), although other methods can be used.

The dimensions, number and density of the areas may vary. In some embodiments, the size of the tissue is 6 mm by 6 mm. Any size of tissue is theoretically possible. In general, the larger the tissue and number of cells, the larger the spatial tag space required. After the sample have been spatially barcoded, various methods can be used to determine the location of the spatial tag. In this step, the skilled artisan understands that many methods can be used to determine the identity and location of the spatial tag. The spatial tag is applied to the whole sample or a region of the sample. This may be done by adding to the sample the spatial tag and any other necessary reagents for aiding binding of the spatial tag to the sample. (e.g., an enzyme such as a polymerase or terminal transfers, as well as any other necessary reagents and cofactors, etc.). Spatial tag can also bind using external stimuli including light, electro, magnetic etc. In this step, the spatial tag will only bind with the sample that have been exposed or not exposed with the stimulus. As such, this step of the method results in targeted immobilization of spatial tags to the sample (e.g., regions of interest or introduce different barcodes or barcode combinations to different regions of the sample).

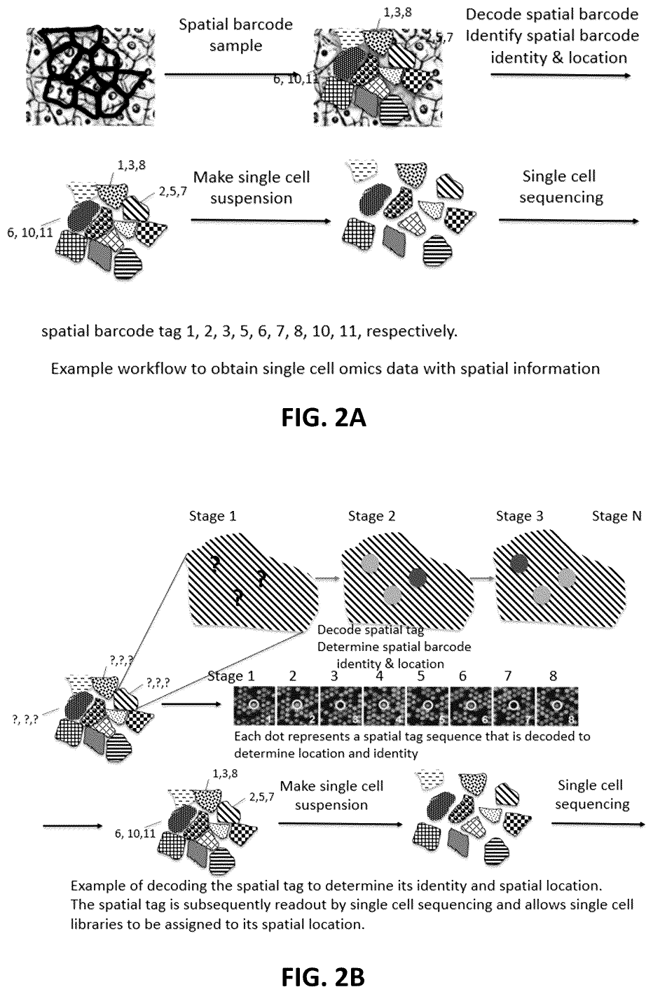

As illustrate in FIG. 1 and FIG. 2, the method results in each cell of the spatial sample differentially labeled with a unique spatial tag or combination of spatial tags, wherein the cells in the different areas have different spatial tags and barcodes. For example, the barcode added to the nucleic acids in area I have a sequence that is distinguishable from the sequences of the barcodes in areas 2-12, and so on. As such, sequences of the barcoded nucleic acids from areas 2-12 can be mapped to a particular area on the sample by the barcode.

After removing any unreacted or unbound spatial tags (i.e., any nucleic acid with a unique barcode), by, e.g., washing the unbound spatial tag from the sample, degrading unbound spatial tag or inactivating the unbound spatial tag, addition of spatial tags and washing steps may be repeated one or more times to produce spatially addressed barcodes that are bound in or on the cells of the sample, or attached to nucleic acid molecules that are in or on the cellular sample. Illustrated by example, if millions of spatial tags of a complex pool of barcodes are bound to the sample, then a subset of hundreds of spatial tags are bound to areas of the sample. In this example, at the end of the spatial barcoding, each area of the cellular sample has obtained a unique set of spatial tags. As would be apparent, different spatial tags (i.e., barcode 1, barcode, etc.) are randomly distributed over the sample, each spatial tag or combination thereof tagging each cell uniquely. At this time, the identities and the location of the spatial tags are unknown.

FIG. 2 illustrates this principle of how the location and identity of the spatial barcodes can be determined using this method. In this example, different spatial tags are produced in a spatial area containing cells. In practice, the spatial tag pool may be more complex (e.g., in the range of millions, trillions of unique spatial tags) and more or less spatial tags per area of a single cell may be used. In some embodiments, one or more spatial tags are tagging a single cell in or on the sample. In some embodiments, the number of spatial tags for each cell in or on the sample is different. In some embodiments, the combination of spatial tags for each cell are different. In some embodiments, cells can have the same spatial tag but the combination of spatial tags for each cell is different.

The cellular sample comprising spatial tags may be made in a variety of different ways. Example systems and methods to tag cells in a tissue sample with spatial tags, so that spatial reconstruction of cell locations within a tissue can be achieved after tissue disaggregation is described in U.S. Patent No. WO 2020/106966 A1 Spatial Mapping of Cells and Cell Types in Complex Tissues, the entire disclosure of which, except for any definitions, disclaimers, disavowals, and inconsistencies, is incorporated herein by reference. The systems and methods described can be combined with single cell expression analysis to correlate cell types with cell location within a structure, such as a tumor. Additionally, the methods and compositions described in current application can be combined with other spatial analysis methods for analyzing macromolecules (i.e. proteins, peptides, polypeptides etc.) as outlined in US20220235405A 1 Methods and Related Kits for Spatial Analysis, the entire disclosure of which, except for any definitions, disclaimers, disavowals, and inconsistencies, is incorporated herein by reference.

In some embodiments, the sample may be made by specific binding of spatial tag(s) on or in the cells. In these embodiments, the spatial tags may bind directly to an endogenous nucleic acid or copy of the same, or indirectly to an endogenous nucleic acid or copy of the same. Alternatively, the spatial tag can bind to the sample in a non-specific manner. In these embodiments, the spatial tag may bind to cell walls, cellular structures, membranes, analytes or derivatives thereof, and other molecular or macromolecular structures. In addition, a spatial tag could be bound to the sample via a binding moiety, e.g., an antibody, aptamer, intercalator, cellular membrane binder, peptide, oligonucleotide probe etc. Oligonucleotide may be linked to binding agents using any convenient method (see, e.g., Gong et al., Bioconjugate Chem. 2016 27: 217-225 and Kazane et al. Proc Natl Acad Sci 2012 109: 3731-3736). Such conjugates have been used for the multiplexed analysis of cellular samples (see Samusik et al Cell 2018 174: 968-981.e15) and can be readily adapted herein. In this latter embodiment, the oligonucleotides may be linked to protein binding agents and, as such, the present method may be used analyze protein epitopes that are on or in a cell. Alternatively, the spatial tag could be by (reversibly) crosslinking to the sample or molecules in or on the sample. For example, the spatial tag can contain moieties (e.g., amine, NHS-ester etc.) that enable crosslinking of the barcode to the sample. In some embodiments, the spatial tag can be permanently or temporarily immobilized in or on the sample. The spatial tag can be administered under limited dilution conditions to ensure that a certain number of spatial tag molecules are immobilized per sample or spatial area e.g., a cell. The number of spatial tag molecules can also be modulated through competitive binding of an inactive molecule and spatial tag to the same affinity partner e.g. spatial tag without an assay handle (‘inactive’) or without a barcode (‘inactive’). The number of spatial tags per sample/cell can be empirically determined and can be balanced against the number of single cell molecules for that cell. As such, the ratio between spatial tag and single cell analytes for that cell can be tuned. In the preferred embodiment, the number of spatial tag molecules are sufficient for the accurate determination of the spatial location at the decoding and single cell assay step, while maximizing sensitivity and detection of single cell analytes during single cell sequencing.

In some embodiments, the spatial tag may contain binding elements to enable binding e.g., oligonucleotide, intercalators, groups that facilitate binding to membranes, hydrophilic or hydrophobic groups that bind to cellular molecules, cellular compartments, affinity agents to analytes (specific or non-specific oligo tag, antibody, etc.) or cellular structures. In any circumstance, the spatial tags are randomly distributed to the cellular samples and the exact location or identity at the time of binding is not known.

In some embodiments, the spatial tag is added through addition. The addition may be non-templated (meaning that there is no underlying nucleic acid “template”) for the addition. These embodiments may be catalyzed by enzymes (e.g., a terminal transferase (for the 3′ end), via chemical addition (for the 3′ or 5′ end), or chemical reaction with molecules in or on the cell. For example, the spatial barcode tag can be added through primer extension using reverse transcription of a poly A primer on an mRNA template. The primer can contain a spatial tag. In some embodiments, only a fraction of the primers extending contain a spatial tag. In some of the embodiments, this spatial tag addition step to the sample is part of the single cell sequencing process performed at a later point in time.

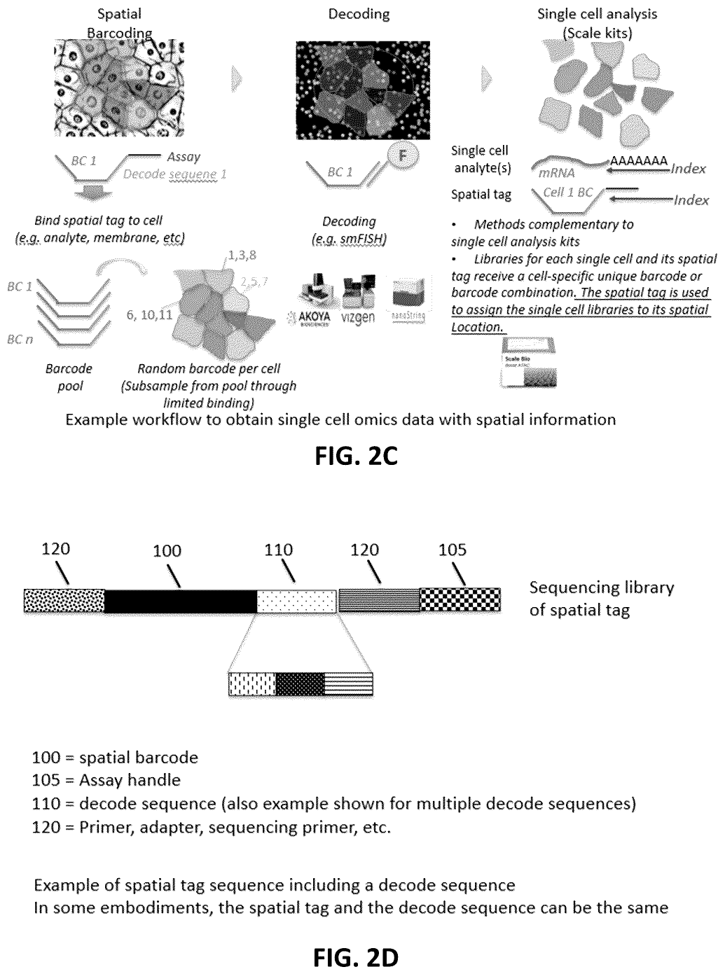

In alternative embodiments, the addition step may be templated (meaning that there may be an underlying nucleic acid “template”. These embodiments may be implemented by a polymerase or ligase. FIG. 3 shows an example of sequencing library configuration for the single cell sequencing libraries for both the RNA library and the spatial tag library. e. As illustrated in FIGS. 1 and 2, this embodiment of the method may comprise binding a high-complexity pool of spatial tags to the sample via non-specific binding (i.e., an oligonucleotide that has a 3′ end common sequence, a 5′ common sequence, and a barcode between the common sequences). The spatial tags are randomly distributed, with each cell receiving a unique combination. The chance that each cell in the sample is labeled uniquely is determined by the complexity of the spatial tag pool, and the number of spatial tags per cell. The larger the complexity of the pool, the higher the chance that each cell would have a unique set of barcodes. Each barcode in the pool can be unique. In some embodiments, multiples of the same barcode can be present. Oligonucleotide pools with many different oligonucleotides and barcodes can be synthesized through split-pool synthesis (e.g. multi-line split DNA synthesis: a novel combinatorial method to make high quality peptide libraries Tabuchi et al. BMC Biotechnology vol. 4, Article number: 19 (2004), >1016 oligonucleotide diversity) or chip-based silicon-based DNA synthesis platform capable of producing over a million unique ssDNA oligos in a single run (e.g. Twist Biosciences). In these embodiments, the spatial tag has an anchor sequence to which a primer can hybridize during single cell barcoding. The anchor sequence (‘assay handle”) that both the spatial tag and the analytes from the same cell receive the same single cell barcode or cell origination barcode (reference to Nolan et al) After spatial barcoding, the identity and location of the barcodes are determined, termed the ‘decoding process’. The artisan known in the field understands that many different approaches and methods can be used. Example of decoding of random arrays has been described: Decoding Randomly Ordered DNA Arrays Genome Res. 2004 May; 14(5): 870-877. Other methods that can be used are: Multispectral imaging systems and method, U.S. Pat. No. 10,964,001, Semin Immunopathol 2023 January; 45(1): 145-157, doi: 10.1007/s00281-022-00974-0. Highly multiplexed spatial profiling with CODEX: bioinformatic analysis and application in human disease. Color-Changing Fluorescent Barcode Based on Strand Displacement Reaction Enables Simple Multiplexed Labeling Koki Makino et al. J. Am. Chem. Soc. 2022, 144, 4, 1572-1579. Systems and methods for determining nucleic acids patent number U.S. Pat. No. 11,098,303B2 incorporated herein. Systems and methods for imaging or determining spatial tags, for instance, within cells. In some embodiments, a plurality of spatial tags may be applied to a sample, and their identity and location within the sample determined, e.g., using fluorescence or multi-color imaging, to determine locations of the spatial tag. A relatively large number of different spatial tags may be identified using a relatively small number of labels or steps, e.g., by using various combinatorial approaches. In some embodiments, labels are quantum dots (QDs), semiconductor nanocrystals, or quantum dots as fluorescent labels, particles a few nanometers in size can be used for visualizing spatial tags (for example, quantum dot imaging platform for single-cell molecular profiling Zrazhevskiy et al. Nature Communications vol. 4, 1619 (2013).

In one embodiment, decoding of the spatial tags occurs through sequential hybridization of fluorescent decoders, imaging, removing the fluorescent decoders, and repeating the process multiple times with different sets of decoders to determine the identity and location of the spatial tags. Technologies related to single molecule FISH can also be used for the detection and identification of the spatial tags e.g., single-molecule Fluorescence in situ Hybridization (smFISH) for RNA Detection in Adherent Animal Cells Haimovich et al. Bio Protoc. 2018 Nov. 5; 8(21). In some embodiments, z-probes, pre-amplifiers or amplifiers, amplification schemes, can be used to visualize and detect the spatial tags e.g. using single molecule mRNA fluorescent in situ hybridization (RNA-FISH) to quantify mRNAs in individual murine oocytes and embryos Xie et al. Scientific Reports volume 8, Article number: 7930 (2018) and references cited herein. In some embodiments, not all spatial tags present in the sample are decoded. The combination of all decoded spatial tags is unique or substantially unique from cell to cell. Other methods of introducing spatial tags including various methods of cell hashing and analyte tagging as previously described (Hashing with barcoded antibodies enables multiplexing and doublet detection for single cell genomics, Stoeckius et al. Genome Biology vol. 19, 224 (2018) or Comparative analysis of antibody-and lipid-based multiplexing methods for single-cell RNA-seq Mylka et al. Genome Biology volume 23, 55 (2022) and reference cited herein). Various spatial methods have been described and can be used for the visualization and identification of the spatial tags described in this invention (for example, see Museum of spatial transcriptomics Moses et al. Nature Methods volume 19, pages 534-546 (2022)). For example, the nCounter Analysis System from nanostring can detect 800+ target analytes (see, Goytain et al. NanoString nCounter Technology: High-Throughput RNA Validation Methods Mol Biol 2020; 2079: 125-139.

After the spatial tag identity and location has been determined, the sample is dissociated into individual cells. A skilled artisan understands that many protocols are available for processing single cells e.g. Tissue dissociation for single-cell and single-nuclei RNA sequencing for low amounts of input material, Wiegleb et al. Frontiers in Zoology volume 19, 27 (2022), Systematic assessment of tissue dissociation and storage biases in single-cell and single-nucleus RNA-seq workflows Denisenko, et al.

Genome Biology volume 21, 130 (2020). In one embodiment, single cells or nuclei from solid tissues are dissociated with the Singulator™ Automated Tissue Dissociation Platform S2 genomics). Dissociation protocols and methods need to be compatible with the spatial tags in a way that the spatial tag stays associated with the single cell or Nuclei.

After sample dissociation, the individual cells or nuclei can be processed using a variety of single cell analysis methods e.g. New frontiers in single-cell genomics Navin et al. (Genome Res. 2021 31: ix-x). Example single cell analysis technologies are described in exponential scaling of single-cell RNA-seq in the past decade Svensson et al. Nature Protocols volume 13, p599-604 (2018). Single-cell sequencing enables the elucidation of RNA, DNA and or protein analytes at single cell resolution. Methods compartmentalize single cells or nuclei in single compartments and assign cell-specific barcodes to each cell to label them uniquely. Alternatively, single cell combinatorial indexing methods analyze each single cell through a split-pool indexing protocol, where cells or nuclei are passed through a unique combination of barcoded wells to label each cell uniquely e.g., comprehensive single-cell transcriptional profiling of a multicellular organism. See Cao et al. 2017, vol 357, Issue 6352, pp. 661-667.

The single cell readout of the current method can range from epigenetics, genomics, proteomics, and multi-omics e.g. Integrative single-cell analysis Stuart et al. Nature Reviews Genetics volume 20, pages 257-272 (2019). Many single assays have been developed including RNA-seq, ATAC-seq, RNA+ATACseq, Multi-seq, Ab-seq, CITE-seq etc. Exemplary assays include the following: 2b-RAD Wang S et al. (2012) Nat Methods 9: 808-810 3C Duan Z. et al. (2012) Methods 58: 277-288 4-C Zhao Z. et al. (2006) Nat Genet 38: 1341-1347 5-C Dostie J. et al. (2007) Nat Protoc 2: 988-1002 Aba-seq Sun Z. et al. (2013) Cell Rep 3: 567-576 ATAC-Seq Buenrostro J. D. et al. (2013) Nat Methods 10: 1213-1218 BisChIP-Seq Statham A. L. et al. (2012) Genome Res 22: 1120-1127 Bisulfite-seq Berman B. P. et al. (2012) Nat Genet 44: 40-46 BLESS Crosetto, N. et al. Nat. Methods 10, 361-365 (2013). Break-seq Hoffman E. A. et al. (2015) Genome Res 25: 402-412 BSAS Masser D. R. et al. (2013) Epigenetics Chromatin 6: 33 BSPP Deng J. et al. (2009) Nat Biotechnol 27: 353-360 BS-Seq Lister R. et al. (2009) Nature 462: 315-322 Bubble-Seq Mesner L. D. et al. (2013) Genome Res 23: 1774-1788 CAB-Seq Lu X. et al. (2013) J Am Chem Soc 135: 9315-9317 caMAB-seq Wu H et al. (2016) Nat Protoc 11: 1081-100 CAP-seq Illingworth R. S. et al. (2010) PLoS Genet 6: e1001134 Capture-C Hughes J. R. et al. (2014) Nat Genet 46: 205-212 CATCH-IT Deal R. B. et al. (2010) Science 328: 1161-1164 Chem-Seq Anders L. et al. (2014) Nat Biotechnol 32: 92-96 Chia-PET Li G. et al. (2010) Genome Biol 11: R22 ChIP-BS-seq Brinkman A. B. et al. (2012) Genome Res 22: 1128-1138 ChIP-exo Yen K. et al. (2013) Cell 154: 1246-1256 ChIPmentation Schmidl C et al. (2015) Nat Methods 12: 963-5 ChIP-Seq Barski A. et al. (2007) Cell 129: 823-837 CPT-seq Amini S. et al. (2014) Nat Genet 46: 1343-1349 DamID Vogel M. J. et al. (2007) Nat Protoc 2: 1467-1478 ddRADseq Peterson B. K. et al. (2012) PLoS One 7: e37135 Digenome-seq Kim D et al. (2015) Nat Methods 12: 237-43 DIP-seq Shen L. et al. (2013) Cell 153: 692-706 Dnase I SIM Cumbie J S et al. (2015) Plant Methods 11: 42 DNaseI-Seq Hesselberth J. R. et al. (2009) Nat Methods 6: 283-289 DNase-Seq Boyle A. P. et al. (2008) Cell 132: 311-322 Drop-ChIP Rotem A et al. (2015) Nat Biotechnol 33: 1165-72 DR-Seq Dey SS et al. (2015) Nat Biotechnol 33: 285-9 DSB-seq Baranello L et al. (2014) Int J Mol Sci 15: 13111-22 Duplex-Seq Schmitt M. W. et al. (2012) Proc Natl Acad Sci USA 109: 14508-14513 EC-seq Parkinson N. J. et al. (2015) Genome Res 25: 226-234 EpiRADSeq Schield D R et al. (2016) Methods in Ecology and Evolution 7: 60-69 FAIRE-Seq Gaulton K. J. et al. (2010) Nat Genet 42: 255-259 fCAB-Seq Song C. X. et al. (2013) Cell 153: 678-691 fC-CET Xia B et al. (2015) Nat Methods 12: 1047-50 fC-Seal Song C. X. et al. (2013) Cell 153: 678-691 FiT-Seq Cejas P et al. (2016) Nat Med G&T-seq Macaulay I. C. et al. (2015) Nat Methods 12: 519-522 G4-seq Chambers V. S. et al. (2015) Nat Biotechnol 33: 877-881 GUIDE-seq Tsai S Q et al. (2015) Nat Biotechnol 33: 187-97 HELP-Seq Oda M. et al. (2009) Nucleic Acids Res 37: 3829-3839 Hi-C Lieberman-Aiden E. et al. (2009) Science 326: 289-293 Histone meth. Barski A. et al. (2007) Cell 129: 823-837 HiTS-Flip Nutiu R. et al. (2011) Nat Biotechnol 29: 659-664 hMeDIP-seq Xu Y et al. (2011) Mol Cell 42: 451-64 HT-ChIP Blecher-Gonen R. et al. (2013) Nat Protoc 8: 539-554 HTGTS Chiarle, R. et al. (2011) Cell 147, 107-119 HT-SELEX Jolma A. et al. (2010) Genome Res 20: 861-873 HydEn-seq Clausen A. R. et al. (2015) Nat Struct Mol Biol 22: 185-191 hyRAD Suchan T et al. (2016) PLoS One 11: e0151651 Ig-Seq Vollmers C. et al. (2013) Proc Natl Acad Sci USA 110: 13463-13468 IMS-MDA Seth-Smith H. M. et al. (2013) Nat Protoc 8: 2404-2412 INSeq Goodman A L et al. (2009) Cell Host Microbe 6: 279-89 JBP1-seq Cui L. et al. (2014) Genomics LAM-HTGTS Hu J et al. (2016) Nat. Protocols 11: 853-871 MAB-seq Wu H. et al. (2014) Nat Biotechnol MAF Khan T A et al. (2016) Sci Adv 2: e1501371 MAINE-Seq Ponts N. et al. (2010) Genome Res 20: 228-238 MALBAC Zong C. et al. (2012) Science 338: 1622-1626 MBDCap-Seq de Assis S. et al. (2012) Nat Commun 3: 1053 MBD-Seq Nair S. S. et al. (2011) Epigenetics 6: 34-44 MDA Dean F. B. et al. (2001) Genome Res 11: 1095-1099 MeDIP-Seq Down T. A. et al. (2008) Nat Biotechnol 26: 779-785 MethylCap-Seq Brinkman A. B. et al. (2010) Methods 52: 232-236 Methyl-seq Brunner A. L. et al. (2009) Genome Res 19: 1044-1056 MIDAS Gole J et al. (2013) Nat Biotechnol 31: 1126-32 MiGS Serre D. et al. (2010) Nucleic Acids Res 38: 391-399 MINCE-seq Ramachandran S et al. (2016) Cell 165: 580-92 Mint-ChIP van Galen P et al. (2016) Mol Cell 61: 170-80 MIPSTR Carlson K. D. et al. (2015) Genome Res 25: 750-761 MIRA Rauch T. A. et al. (2010) Methods 52: 213-217 MNase-Seq Schones D. E. et al. (2008) Cell 132: 887-898 MPE-seq Ishii H et al. (2015) Proc Natl Acad Sci USA 112: E3457-65 MRE-Seq Maunakea A. K. et al. (2010) Nature 466: 253-257 NG Capture-C Davies J O J et al. (2016) Nat Meth 13: 74-80 NOME-Seq Han H. et al. (2011) Hum Mol Genet 20: 4299-4310 NSCR Kunnev D. et al. (2015) Genome Res 25: 558-569 NS-seq Foulk M S et al. (2015) Genome Res 25: 725-Nucleo-Seq Valouev A. et al. (2011) Nature 474: 516-520 nuc-seq Wang Y. et al. (2014) Nature 512: 155-160 ORGANIC Zentner G. E. et al. (2013) PLoS Genet 9: e1003317 OS-Seq Myllykangas S. et al. (2011) Nat Biotechnol 29: 1024-1027 oxBS-Seq Booth M. J. et al. (2012) Science 336: 934-937 PAT-ChIP Fanelli M et al. (2011) Nat Protoc 6: 1905-19 PBAT Miura F. et al. (2012) Nucleic Acids Res 40: e136 PB-seq Guertin M. J. et al. (2012) PLoS Genet 8: e1002610 PD-Seq Arango D. et al. (2013) Proc Natl Acad Sci USA 110: E2153-2162 PDZ-Seq Ernst A. et al. (2010) Mol Biosyst 6: 1782-1790 PE RAD-Seq Willing E. M. et al. (2011) Bioinformatics 27: 2187-2193 ProP-PD Ivarsson Y. et al. (2014) Proc Natl Acad Sci USA 111: 2542-2547 Pu-seq Daigaku Y. et al. (2015) Nat Struct Mol Biol 22: 192-198 RAD Baird N. A. et al. (2008) PLoS One 3: e3376 Rapture Ali O A et al. (2016) Genetics 202: 389-400 RBBS Meissner A et al. (2008) Nature 454: 766-70 RC-Seq Baillie J. K. et al. (2011) Nature 479: 534-537 RedBS-Seq Booth M. J. et al. (2014) Nat Chem 6: 435-440 Repli-Seq Hansen R. S. et al. (2010) Proc Natl Acad Sci USA 107: 139-144 Rep-Seq Benichou J. et al. (2012) Immunology 135: 183-191 Ribose-seq Koh K. D. et al. (2015) Nat Methods 12: 251-257 RRBS Meissner A. et al. (2008) Nature 454: 766-770 RRMAB-seq Neri F. et al. (2015) Cell Rep 10: 674-683 Safe-SeqS Kinde I et al. (2011) Proc Natl Acad Sci USA 108: 9530-5 scABA-seq Mooijman D et al. (2016) Nat Biotechnol advance online publication scATAC-seq Cusanovich D A et al. (2015) Science 348: 910-4 (Cell index) scATAC-seq Buenrostro J. D. et al. (2015) Nature 523: 486-490 (Microfluidics) scBS-seq Smallwood S. A. et al. (2014) Nat Methods 11: 817-820 scChIP-seq Rotem A et al. (2015) Nat Biotechnol 33: 1165-72 scM&T-seq Angermueller C et al. (2016) Nat Methods advance online publication scMT-Seq Hu Y et al. (2016) Genome Biol 17: 88 scRC-Seq Upton K R et al. (2015) Cell 161: 228-39 scRRBS Guo H. et al. (2015) Nat Protoc 10: 645-659 scTrio-seq Hou Y et al. (2016) Cell Res 26: 304-19 SELEX Jolma A. et al. (2010) Genome Res 20: 861-873 SELEX-seq Slattery M. et al. (2011) Cell 147: 1270-1282 SLAF-Seq Sun X et al. (2013) PLoS One 8: e58700 SMDB Lan F et al. (2016) Nat Commun 7: 11784 smMIP Hiatt J. B. et al. (2013) Genome Res 23: 843-854 SNES Leung M. L. et al. (2015) Genome Biol 16: 55 Sono-Seq Auerbach R. K. et al. (2009) Proc Natl Acad Sci USA 106: 14926-14931 SSB-Seq Baranello L et al. (2014) Int J Mol Sci 15: 13111-22 TAB-Seq Yu M. et al. (2012) Cell 149: 1368-1380 TAmC-Seq Zhang L. et al. (2013) Nat Commun 4: 1517 TC-Seq Klein I. A. et al. (2011) Cell 147: 95-106 THS-seq Sos BC et al. (2016) Genome Biol 17: 20 TN-Seq van Opijnen T. et al. (2013) Nat Rev Microbiol 11: 435-442 T-WGBS Wang Q. et al. (2013) Nat Protoc 8: 2022-2032 UMI-4C Schwartzman O et al. (2016) Nat Methods advance online publication WGBS Lister R. et al. (2009) Nature 462: 315-322 X-ChIP-seq Skene P. J. et al. (2014) Elife 3: e02042.

One aspect of this invention is that both the spatial tag tags and the analytes can be efficiently labeled with single cell specific barcode or cell originating barcodes (See, e.g., WO2012106385A2, which is incorporated by reference in its entirely) and converted into single cell sequencing libraries.

Many methods and technologies are currently available to perform single cell analysis (see, e.g. a comprehensive review Exponential scaling of single-cell RNA-seq in the past decade Svensson et al. Nature Protocols volume 13, pages 599-604 (2018) and references cited herein). Single-cell sequencing examines the sequence information from individual cells with next-generation sequencing technologies, providing a high-resolution readout of analytes of individual single cells (RNA, DNA, cDNA, epigenomics, protein etc.). Many commercial companies make instrument or instrument-free products for the analysis of single cells including Scale Biosciences, 10× Genomics, BD Biosciences, Takara Biosciences, Bio-Rad, Parse Biosciences. Single cells are tagged with a unique barcode or barcode combinations resulting in barcoded sequencing libraries of the cells to be assigned to each individual cell. Single cells are isolated in droplets, wells, or unique barcoded through split-pool or combinatorial barcoding as described by Svensson et al. and comprehensive single-cell transcriptional profiling of a multicellular organism Cao at al. Science 2017 Vol 357, Issue 6352 pp. 661-667, Nat Protoc. 2023, 188-207 optimized single-nucleus transcriptional profiling by combinatorial indexing Martin et al. or related methods. Any of the current single cell analysis methods can be utilized for the methods and compositions described in this invention.

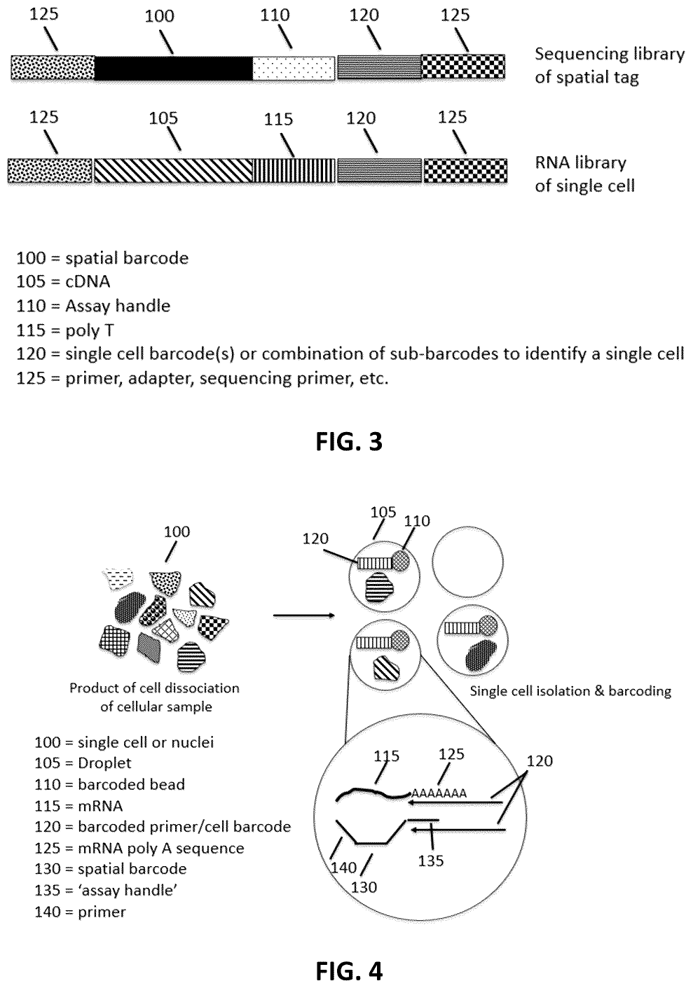

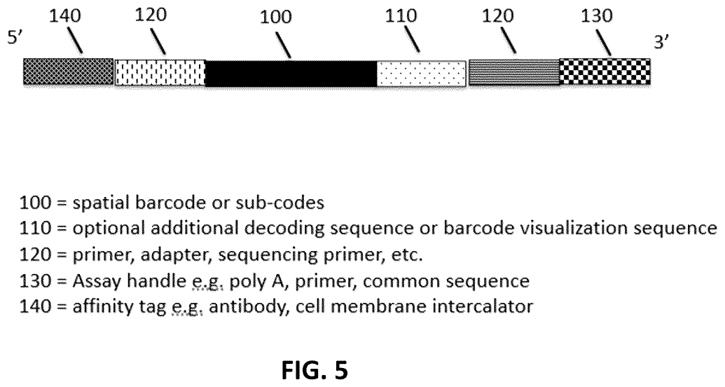

Examples of spatial single cell analysis using spatial tags and single cell sequencing are described below. In some embodiments, the spatial tag contains an oligonucleotide sequence (‘assay handle’) in order to make sequencing libraries of the spatial tag together with the preparation of single cell sequencing libraries of the analytes of the same cell. FIG. 4 describes an example workflow. In this example, the sample is exposed to a population of spatial tags, each spatial tag binding randomly to, for example, the cellular membrane of cells through a peptide affinity tag (for example, Mylka, Genome Biology volume 23, Article number: 55 (2022). Non-bound spatial tags are removed by washing the sample. Subsequently, the spatial tags are stained with Z-probes and pre-amplification and amplification mix probes to enhance the signal in the decoding process (for example, Fang Xie, Scientific Reports vol. 8, 7930 (2018) either through specific binding to spatial tag or an aiding oligonucleotide sequence). The specific spatial tags are detected and identified using sequential hybridization and de-hybridization procedure as outlined in U.S. Pat. No. 11,098,303 Systems and methods for determining nucleic acids or Decoding Randomly Ordered DNA Arrays Genome Res. 2004, 14(5): 870-877. Once the identity and location of each spatial tag is determined, the sample is dissociated into single cells or nuclei, each containing a unique spatial tag or spatial tag combination. Single cells and the associated spatial tags are labeled with cell-specific barcodes, sequencing libraries are generated, and single cell libraries are sequenced. Sequencing reads with the same cell barcode are grouped together and for each single cell the spatial tag is determined (the single cell sequencing libraries of the analyte(s) of interest and the spatial tag for each cell have the same single cell barcode). Once the relationship between the single cell sequencing libraries and the spatial tag has been assigned and established the single cell sequencing reads can be assign to its spatial coordinates of the sample.

In some embodiments, the spatial tag can contain a primer or sequence to facilitate in situ or in vitro amplification. Various amplification schemes can be envisioned including PCR amplification, rolling circle amplification (RCA), and concatenation amplification schemes (e.g., Hybridization chain reactions), multiple displacement amplification and the like. This rolling circle amplification reaction is described in the discovery of rolling circle amplification and rolling circle transcription Mohsen et al. Acc. Chem. Res. 2016, 49, 11, 2540-2550. In some embodiments, the spatial tag may contain a primer that initiates RCA amplification. Hyperbranched hybridization chain reaction for triggered signal amplification and concatenated logic circuits Sai Bi et al. Angewandte chem. 2015. In some embodiments, the spatial tag or decode sequence is a circular sequence that can be amplified.

In some embodiments, the spatial tag needs to be in situ amplified before decoding or before or during single cell sequencing to ensure the spatial tag is observed. In the preferred embodiment, the spatial location of the spatial tag is maintained during amplification. In some cases, in vitro amplification of the spatial tag for single cell sequencing can occur in a compartment (droplet, well, embedded bead e.g., polymer bead). In some embodiments, the spatial tag provides the cell specific barcode in the compartment used for single cell sequencing. In some examples, each spatial tag or set of spatial tags associated with a single cell/nuclei is amplified inside a compartment (droplet, well, microwell etc.) and used as the barcoding primer for the single cell sequencing libraries (see for example, US20220325275, herein incorporated by reference). In some embodiments, the spatial tag is locally amplified and locally distributed. In some embodiments, the spatial tag or amplification product can bind target molecules of interest. In some embodiments multiple combinatorial spatial hash tags will be concatenated and attached to the cellular analytes.

The decoding process, the identification of the identity of the spatial tag and location of the tag can be performed using microscopy. In some embodiments, the process of decoding involves staining the spatial tag or proxy sequence or amplicons/amplification products thereof with decoders (e.g., fluorescent oligonucleotides), washing unbound decoder, imaging (stage 1), and repeating the process with decoding and imaging using multiple stages (See FIG. 6). In some embodiments, the same spatial tag is stained with a decoder and imaged at the same location multiple times with different decoding pools to determine the identify and position of the spatial tag. Super-resolution microscopy (SRM) can be used that encompasses fluorescence imaging techniques with the capability to resolve objects below the classical diffraction limit of optical resolution. In some embodiments, the resolution can be <100 nm. In some embodiments, the imaging can be performed in multiple dimensions, e.g. 3D. 3D imaging has the advantage that the spatial tag can be identified in a 3D format enabling 3D spatial single cell analysis (For example, Wang et al. three-dimensional intact-tissue sequencing of single-cell transcriptional states and US20210164039 herein both documents incorporated by reference).

In some embodiments, the spatial tag is diffused through the sample to spatially tag cells across the tissue and sample. The spatial tag can be diffused or actively transported using osmosis, electrophoresis, magnetism, or other means. Confocal, structured illumination and other luminescence and fluorescence techniques can be used. In some embodiments, the imaging is automated. An example imaging system is described here with <50 nm resolution and 1-2 copies of molecules detection sensitivity high-plex multi-omic analysis in FFPE at subcellular level by spatial molecular imaging He et al. Biorxiv 2022.

In some embodiments, the polymer embedded tissue may be subjected to expansion, stretching, or enlargement during the process of determining the spatial barcode location and identity. Expansion microscopy (ExM) is a technique to overcome the diffraction limit of light microscopy that can be applied in both tissues and cells. In ExM, samples are embedded in a swellable polymer gel to physically expand the sample and isotopically increase resolution in x, y, and z. In some embodiments, the expansion is only used during spatial barcode location detection and identification, and spatial information is directly recorded during time of expansion. Methods and compositions of expansion in the context of spatial single cell analysis have been described in the literature (Alon et al., Science 371, 481, Expansion microscopy: principles and uses in biological research Wassie et al. Nature Methods vol. 16, pages 33-41 (2019), Visualizing cellular and tissue ultrastructure using Ten-fold Robust Expansion Microscopy (TREx) Damstra et al. Elife 2022 and US20210190652 herein incorporated by reference). In some embodiments, the embedding of the tissue to facilitate expansion is reversible and is compatible with down-stream single cell analysis.

In addition to the labeling methods described above, the sample or spatial tag may be stained using a cytological stain, either before, during, or after performing the method described above. In these embodiments, the stain may be, for example, phalloidin, gadodiamide, acridine orange, Bismarck brown, barmine, Coomassie blue, bresyl violet, brystal violet, DAPI, hematoxylin, eosin, ethidium bromide, acid fuchsine, haematoxylin, Hoechst stains, iodine, malachite green, methyl green, methylene blue, neutral red, Nile blue,

Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead (II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain and/or spatial tag may be specific for any feature of interest, such as a protein or class of proteins, phospholipids, DNA (e.g., dsDNA, ssDNA), RNA, an organelle (e.g., cell membrane, mitochondria, endoplasmic recticulum, golgi body, nuclear envelope, and so forth), or a compartment of the cell (e.g., cytosol, nuclear fraction, and so forth). The stain may enhance contrast or imaging of intracellular or extracellular structures. In some embodiments, the sample may be stained with haematoxylin and eosin (H&E). In these embodiments, the sample may be analyzed by microscopy to produce one or more images of the sample, prior to, during, or after adding spatial tags to the sample.

In some embodiments, the cellular sample is contained in a flow cell or microfluidic device to aid reagent addition (e.g., spatial tags etc.) and wash steps. In some embodiments, the flow cell is sub-divided into multiple sections enabling the delivery of 2 or more different reagents and solutions, imaging cycle. Example figure https://medical-technology.nridigital.com/medical_technology_nov19/imt_company_insight.

After the single cell sequencing method described above, the single cell barcoded analyte nucleic acids are collected, sequenced en masse, and the single cell analyte sequences mapped to a spatial location in the sample using the correlation between the single cell barcodes and the spatial tags. The single cell barcode serves as a spatial address for the analyte sequence via correlation with the spatial tag. In some embodiments, the sequences from a particular cell in the sample can be resolved from sequences from other cells in the sample. In these embodiments, the method may further comprise sequencing the barcodes produced and at least part of the nucleic acid molecules to which they are attached, or an amplification product thereof, and mapping the sequenced nucleic acid molecules to a site in or on the cellular sample using the barcode to which it is attached. The barcoded nucleic acids can have PCR primer binding sites, thereby facilitating amplification of the barcoded nucleic acids. Alternatively, the barcoded nucleic acids may have an affinity tag, thereby facilitating their enrichment. In some embodiments, a subset of the barcoded nucleic acids may be enriched and sequenced, e.g., by enriching for barcoded nucleic acids that have a particular barcode, or nucleic acid sequence.

The barcoded nucleic acids including the barcoded single cell libraries and barcoded spatial tags may be sequenced by any suitable system Illumina's reversible terminator method, Roche's pyrosequencing method (454), Life Technologies' sequencing by ligation (the SOLID platform), Life Technologies' Ion Torrent platform or Pacific Biosciences' fluorescent base-cleavage method and any other platforms e.g. Oxford Nanopore. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10: 609-18); Fox et al (Methods Mol Biol. 2009; 553: 79-108); Appleby et al (Methods Mol Biol. 2009; 513: 19-39) English (PLoS One. 2012 7: e47768) and Morozova (Genomics. 2008 92: 255-64), which are incorporated by reference for the general descriptions of the methods and the particular steps of the methods, including all starting products, reagents, and final products for each of the steps.

The sequencing step may be done using any convenient next generation sequencing method and may result in at least 10,000, at least 100,000, at least 500,000, at least 1M at least 10M at least 100M, at least 1B or at least 10B sequence reads per reaction. In some cases, the reads may be paired-end reads.

The method may be used to map endogenous nucleic acid molecules. For example, the method may be used to map mRNA sequences. In this example, which is illustrated in FIG. 3, cDNA is synthesized in situ using a tailed primer, and spatial tags are added to the 5′ ends of the cDNA molecules using a splint template using the method described above. In this example, first strand cDNA may be collected, second strand cDNA may be produced, and the second strands amplified by PCR and then sequenced. The barcodes appended to the cDNA allow the cDNA sequences to be mapped to a site on the sample.

In addition, the method may be used to map endogenous epitopes, which may be extracellular or intracellular. In these embodiments, a binding agent, e.g., an antibody or aptamer, that is non-covalently (e.g., via a streptavidin/biotin interaction) or covalently (e.g., via a “click” reaction (see, e.g., Evans Aus. J. Chem. 2007 60: 384-395) or the like) linked to a single-stranded reversibly terminated oligonucleotide in a way that the binding agent can still bind to its binding site is used to label the sample. This step may involve contacting the sample (e.g., an FFPE section mounted on a planar surface such as a microscope slide) with all of the binding agents, en masse under conditions by which the binding agents bind to complementary sites (e.g., protein epitopes) in the sample. In some embodiments, the binding agents may be cross-linked to the sample, thereby preventing the binding agents from disassociating during subsequent steps. In some cases, the oligonucleotides may also contain an identifier sequence that identifies the antibody to which it is bound. In these embodiments, the method can be performed using at least 10, at least 50 or at least 100 different antibodies (i.e., antibodies that recognize epitopes on different proteins). The sequences should contain the added barcode as well as the identifier sequences, thereby allowing the binding site for each antibody to be mapped. These embodiments could be used to resolving cellular components and structures. Combining a cellular component barcode (barcode specific to a specific cell component) with spatial tag synthesis provides a platform for spatially resolving components and cellular sub-structures. Intercalating barcodes or oligos or antibody-oligo conjugates against cellular structure(s) can be used to spatially label and barcode components, including, but not limited, organelles, membranes, nuclei, Golgi apparatus, lysosome, peroxisome, pores, ER, centrioles, mitochondria, ribosomes etc. This enables spatial mapping of cellular structures and cellular structures in relation to the analytes. Cellular component barcoding can also serve as a reference marker, outlines the boundaries of a cell in relation to other cells, enables estimation of the size and shape of the cell, enables the measurement of the distance between a component and analyte etc.

The method can be used to map an epigenomics state. Examples include, but not limited, methylation, open-chromatin state (e.g., ATAC-seq), DNA-protein binding etc. Analytes can be pre-processed before barcode synthesis. For example, DNA can be modified with transposon sequences using a process called transposition (e.g., transposases) enabling ATAC-seq, cut-and-tag assays, whole-genome, multi-omics (e.g., ATAC+RNA) etc. The analyte of interest can be DNA, RNA, cDNA, protein, carbohydrate, small molecule, large molecule, drug, or any combination of analytes etc.

The sequencing data may be used to construct an image of the sample in which each barcode essentially becomes a pixel in the image. In some embodiments, the resolution of the image may be down to 1 nm, 10 nm, 100 nm, 1 μm, 10 μm.

In these embodiments, the resulting image can be false colored, where the different colors correspond to different RNAs or epitopes, and the intensity of any color in any single pixel of a cell correlates with the number of sequences reads obtained for the analyte (for example through unique molecular identifiers (UMI) attached to the sequencing library or analyte). In many cases, the image may be superimposed with an image of the sample, stained as described above.

Analysis methods. Various existing and novel analysis methods can be used. For example, deciphering tissue structure and function using spatial transcriptomics Walker, Communications Biology vol. 5, 220 (2022), Squidpy: a scalable framework for spatial omics analysis Palla et al, Nature Methods vol. 19, p171 (2022), and BarWare: efficient software tools for barcoded single-cell genomics Swanson et al and references cited in the papers). In some embodiments, the computation analysis approaches and methods are related to cell hashing, for example, Cell Hashing with barcoded antibodies enables multiplexing and doublet detection for single cell genomics Stoeckius, Genome Biology vol. 19, 224 (2018) or methods and compositions related to Nuclei: Nuclei multiplexing with barcoded antibodies for single-nucleus genomics Gaublomme, Nature Comm. vol. 10, 2907 (2019), BMC Bioinformatics. 2022; 23: 106. In some embodiments, one or more spatial tags are analyzed in either the spatial or single cell sequencing data sets. 2 or more tags provide redundancy, improves accuracy, and in general improves the ability to link the single cell sequencing data to its spatial location.

Example analysis workflow. From the imaging data a set of spatial tags with their respective X-Y (and optionally Z) positions and identity are obtained. The single cell sequencing data provides sequencing reads with the same single cell barcode for both the libraries of the analyte and the matching spatial tags. Each candidate read is matched to the set of spatial tags finding the most likely matching spatial tag as well as any spatial tags it might match (probabilistically). Count the number of reads for each spatial tag in each cell. Obtain a probabilistic estimate of the abundance of each spatial tag in each cell using the full spectrum of read to spatial tag matches (secondary hits). The spatial tag abundance values, along with the observed X, Y (and optionally Z) position of each spatial tag, make the spatial tag signature of the cell. For each cell we now determine the most likely spatial position/area, that explains the observed spatial tag signature e.g., a single real tag, mapping the cell to that position. Multiple spatial tags that are spatially close, mapping to cell to the area with these barcodes. A statistical model that takes as input the spatial tag signature and then computes the statistical likelihood for the cell or originate from position x1,y1, (and optionally z1) given the positions of all spatial tags in its signature. Select either the single most likely position or report a range of statistically likely positions. The model considers background detection of spatial tags, i.e. if the abundance of a spatial tag is low it might be a background observation, and the tag position hence irrelevant to the cell position. If multiple spatial tags are abundant in the cell and the tags are close to each other spatially, we have higher confidence in the position of the cell at those spatial tag sites. If a spatial tag is observed at multiple positions in, for example, the imaging data, only one of those must match the inferred position of the cell. The model might be a mixture model, solved through an expectation-maximization (EM) algorithm.

The methods described herein find general use in a wide variety of applications for analysis of any sample (e.g., in the analysis of tissue sections, sheets of cells, spun-down cells, etc.). Further, the method has a variety of clinical applications, including, but not limited to, diagnostics, prognostics, disease stratification, personalized medicine, clinical trials, and drug accompanying tests.

In particular embodiments, the sample may be a section of any tissue, including skin (melanomas, carcinomas, etc.), soft tissue, bone, breast, colon, liver, kidney, adrenal, gastrointestinal, pancreatic, gall bladder, salivary gland, cervical, ovary, uterus, testis, prostate, lung, thymus, thyroid, parathyroid, pituitary (adenomas, etc.), brain, spinal cord, ocular, nerve, and skeletal muscle, etc. In some embodiments, the sample may be a tissue biopsy obtained from a patient. Biopsies of interest include both tumor and non-neoplastic biopsies of any tissue.

The above-described method can be used to analyze cells from a subject to determine, for example, whether the cell is normal or not or to determine whether the cells are responding to a treatment. In one embodiment, the method may be employed to determine the degree of dysplasia in cancer cells. In these embodiments, the cells may be a sample from a multicellular organism. A biological sample may be isolated from an individual, e.g., from a soft tissue. In particular cases, the method may be used to identify cancer cells in a sample.

In some embodiments, the method may involve obtaining data (an image) as described above (an electronic form of which may have been forwarded from a remote location), and the image may be analyzed by a doctor or other medical professional to determine whether a patient has abnormal cells (e.g., cancerous cells) or which type of abnormal cells are present. The image may be used as a diagnostic to determine whether the subject has a disease or condition, e.g., a cancer. In certain embodiments, the method may be used to determine the stage of a cancer, to identify metastasized cells, or to monitor a patient's response to a treatment, for example.

The compositions and methods described herein can be used to diagnose a patient with a disease. In some cases, the presence or absence of a biomarker in the patient's sample can indicate that the patient has a particular disease (e.g., a cancer). In some cases, a patient can be diagnosed with a disease by comparing a sample from the patient with a sample from a healthy control. In this example, a level of a biomarker, relative to the control, can be measured. A difference in the level of a biomarker in the patient's sample relative to the control can be indicative of disease. In some cases, one or more biomarkers are analyzed to diagnose a patient with a disease. The compositions and methods of the disclosure are particularly suited for identifying the presence or absence of, or determining expression levels, of a plurality of biomarkers in a sample.

In some cases, the compositions and methods herein can be used to determine a treatment plan for a patient. The presence or absence of a biomarker may indicate that a patient is responsive to or refractory to a particular therapy. For example, a presence or absence of one or more biomarkers may indicate that a disease is refractory to a specific therapy, and an alternative therapy can be administered. In some cases, a patient is currently receiving the therapy and the presence or absence of one or more biomarkers may indicate that the therapy is no longer effective.

In some cases, the method may be employed in a variety of diagnostic, drug discovery, and research applications that include, but are not limited to, diagnosis or monitoring of a disease or condition (where the image identifies a marker for the disease or condition), discovery of drug targets (where a marker in the image may be targeted for drug therapy), drug screening (where the effects of a drug are monitored by a marker shown in the image), determining drug susceptibility (where drug susceptibility is associated with a marker) and basic research (where is it desirable to measure the differences between cells in a sample).

In certain embodiments, two or more different samples may be compared using the above methods. The different samples may be composed of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen, etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material contains cells that are susceptible to infection by a pathogen such as a virus, e.g., human immunodeficiency virus (HIV), etc., and the control material contains cells that are resistant to infection by the pathogen. In another embodiment, the sample pair is represented by undifferentiated cells, e.g., stem cells, and differentiated cells. In some embodiments, two or more samples (either different tissue samples or areas of the same sample) can be processed and analyzed simultaneously. Many single cell analysis systems provide input for 2 or more samples. In another embodiment of the invention, the sample may be susceptible to CRISPR editing, editing system, or perturbation. In some embodiments, the perturbation can be a chemical compound, drug etc. The perturbation may be marked with a barcode tag or spatial tag, and the perturbation or edit information can be captured in single cell sequencing (see Cell. 2016, 167(7): 1853-1866.e17. Dixit et al. Perturb-seq: Dissecting molecular circuits with scalable single cell RNA profiling of pooled genetic screens).

The images produced by the method may be viewed side-by-side or, in some embodiments, the images may be superimposed or combined. In some cases, the images may be in color, where the colors used in the images may correspond to the sequences of the nucleic acids.

Cells from any organism, e.g., from bacteria, yeast, plants, and animals, such as fish, birds, reptiles, amphibians, and mammals may be used in the subject methods. In certain embodiments, mammalian cells, i.e., cells from mice, rabbits, primates, or humans, or cultured derivatives thereof, may be used.

In some aspects, the described technologies can include a kit. A “kit” is generally understood as a specific type of package that contains two or more discrete components, and those components work together for a specific purpose, or to achieve a specific result. Kit can include, but not limited, spatial tags, optionally, enzymes or chemicals to attach spatial tags, oligonucleotides, decoders to identify the identity of the spatial tag or a combination thereof.

Provided herein are kits including a spatial tag comprising a barcode sequence, which comprises identifying information regarding the location and identity of the spatial tag; wherein the spatial tag can bind to a macromolecule or cellular component. In some embodiments, the kit comprises a plurality of spatial tags. In some embodiments, the spatial tag comprises an assay handle. In some embodiments, the kit comprises spatial tags, oligonucleotides, wash, and binding buffers, fluorescent or luminescent decoders, and enzymes, optionally some components are provided as a mixture.

1. A method for adding spatial tags to cells, comprising:
(a) distinguishably labeling at least some of the cells of a sample containing spatially associated cells;
(b) analyzing the sample to identify (i) the positions of cells in the sample and (ii) the labels that are bound to the cells; and
(c) dissociating the sample into individual cells without disassociating the tags from the cells, to obtain a cell suspension in which the locations of some of the cells in the sample of (a) is known.

2. The method of claim 1, further comprising:
(d) on a cell-by-cell basis analyzing: (i) the labels associated with associated with the cells of the cell suspension and (ii) analytes that are in or on at least some of the cells in the cell suspension.

3. The method of claim 2, further comprising
(e) mapping data obtained in (d)(ii) to a position of a cell identified in step (b).

4. The method of claim 1, wherein the method comprises:
(a) labeling a sample containing spatially associated cells with a population of nucleic acid tags, wherein at least some of the cells in the sample become distinguishably tagged by the nucleic acid tags;
(b) analyzing the sample to identify:
(i) the positions of cells in the sample; and
(ii) on a cell-by-cell basis, the nucleic acid tags that are bound to the cells of (b)(i); and
(c) dissociating the sample into individual cells without disassociating the tags from the cells, to obtain a cell suspension in which at least some of the cells are distinguishably tagged.

5. The method of claim 4, further comprising:
(d) on a cell-by-cell basis:
(i) analyzing analytes in or on the cells of the cell suspension to produce single cell data; and
(ii) sequencing the nucleic acid tags that are associated with the cells of the cell suspension.

6. The method of claim 5, further comprising:
(e) on a cell-by-cell basis, mapping the single cell data of step (d)(i) to a position in the sample using the tag sequences of (d)(ii).

7. The method of claim 6, wherein:
step (b) further comprises (iii) recording the positions of distinguishably tagged cells in the sample based the result of steps (d)(i) and (d)(ii); and
in step (e) the single cell data for cells that distinguishably tagged are mapped using the positions recorded in step (b)(i).

8. The method claim 1, comprising:
(a) obtaining a cellular sample comprising analytes;
(b) adding spatial tags to the sample, wherein single cells in the sample receive a unique set of spatial tags;
(c) optionally removing any unbound or reacted spatial tag after step (b)
(d) determining the identity of the spatial tag location and identity;
(e) dissociating the tissue into individual cells, nuclei or cellular sub-components while maintaining the association with the spatial tags; and
(f) performing single cell sequencing

9. The method of claim 8, further comprising determining the single cell barcode and associated single cell spatial tag by sequencing the barcoded libraries produced in step (f) and at least part of the nucleic acid molecules to which they are attached, or an amplification product thereof.

10. The method of claim 9, further comprising mapping the sequenced nucleic acid molecules to a site in or on the cellular sample using the spatial tag for each individual single cell.

11. The method of claim 1, wherein the cellular sample is a tissue section.

12. The method of claim 1, wherein the cellular sample of (1a) is obtained by binding or reacting a spatial tag or combination of spatial tags to a sample that contains cells.

13. The method of claim 1 wherein the spatial tag is a pool of barcoded oligonucleotides.

14. The method of claim 1, wherein the cellular sample of (b) is made by
randomly distributing spatial tags on or in a cellular sample such that the majority of cells each receives a unique combination of spatial tags.

15. The method of claim 14, wherein the spatial tags are bound to the sample through non-specific binding.

16. The method of claim 14, wherein the spatial tag is tethered directly or indirectly to a binding agent (e.g., an antibody, aptamer, lipid) in or on the sample.

17. The method of claim 1, wherein the spatial tag is bound directly or indirectly to an analyte.

18. The method any prior claim 1, wherein the binding of the spatial tag is performed enzymatically or chemically.

19. The method of claim 1, wherein the pool of spatial tags is over 1,000 unique spatial tags.

20. The method of claim 1, wherein the pool of spatial tags is over 1,000,000 unique spatial tags.

21-40. (canceled)
